(19) United States
(12) Patent Application Publication
(10) Pub. No.: US 2003/0044838 A1
(43) Pub. Date: Mar. 6, 2003

(54) PEPTIDE LIGANDS THAT BIND TO SURFACES OF BACTERIAL SPORES

(76) Inventor: CHARLES L. TURNBOUGH, HOMEWOOD, AL (US)

Correspondence Address: GLNNA HENDRICKS, PO BOX 2509, FAIRFAX, VA, 220312509

(21) Appl. No.: 09/229,751

(22) Filed: Jan. 14, 1999

Related U.S. Application Data
(60) Provisional application No. 60/071,411, filed on Jan. 14, 1998.

(51) Int. Cl. G01N 33/53; G01N 33/554; G01N 33/569; C07K 16/12

(52) U.S. Cl. 435/7.1; 435/7.32; 530/388.4

(57) ABSTRACT

Peptides which bind to surfaces of bacterial spores have been identified by means of biopanning using phage-displayed peptide libraries. The sequences identified thereby are useful for binding studies to determine presence of spores in the environment or clinical setting.

This application takes priority from Provisional Patent Application 60/071,411 filed Jan. 14, 1998.

This invention was made with government support under the Defense Advance Research Project Agency project number MDA972-96-0003. The government of the United States has certain rights in the invention.

### BACKGROUND OF THE INVENTION

The capture and identification of bacterial spores is useful for detecting pathogenic or otherwise harmful bacteria. Often the presence of spores can indicate to the researcher or epidemiologist the presence of virulent organisms. It is also important to determine the presence of spores of pathogenic organisms in the environment in order to more effectively control spread of infections. The ability to produce a monitorable tag or ligand that will bind specifically to the bacterial spore would provide a valuable tool for identifying pathogenic organisms in the infected patient and in the environment.

The use of phage-displayed peptide libraries to identify peptide sequences that will bind to particular receptors has been used to evaluate the structure of proteins. (See D'Mello, et al, Virology, 237(2): 319-26 (1997) and Salonen, et al, Journal of General Virology 79 (pt4): 659-65 (1998) regarding mapping of antibodies and Marzari, et al, FEBS Lett. 411(1) 27-31 (1997) regarding phage display of B. thuringiensis insecticidal toxin.) A synopsis regarding the use of phage display has been reviewed by Smith and Petrenko in Chemical Reviews, 97(2) 391-410 (1997). In that review, the authors discuss the general usefulness of this procedure for evaluation of proteins, including antibodies. However, none of the above references suggest using phage display peptide libraries for identifying peptide sequences which bind to whole cells.

### SUMMARY OF THE INVENTION

This invention relates to the capture and/or identification of microorganisms and their spores by preparing peptides which bind to the spores. The peptides are included in phage display peptide libraries that are commercially available, and the peptides that bind to spores are identified using biopanning. While peptide sequences which bind to proteins, especially antibodies, have been studied, the method has not been used to identify peptides that bind to whole microorganisms.

The peptides of the invention and the methods by which the useful peptides are identified are disclosed herein. It is possible, using the peptides which bind to the surface of a cell which are generated by methods described herein, to identify the presence of spores of organisms in the environment and in the clinical setting. Using means of the invention, it is also possible to provide means for protecting potential hosts from exposure to disease-causing spores by administration of peptides which bind to the spores.

The peptides of the invention which bind to Bacillus subtilis contain the amino acid sequence Asn-His-Phe-Leu (NHFL) (Seq. ID No. 1). Additional amino acids containing proline, to provide the sequence NHFLP (Seq. ID No. 39) are particularly preferred sequences.

Peptides of the invention which bind to Bacillus anthracis have the sequence Thr-Ser-Gln-Asn-Val-Arg-Thr (TSQNVRT) (Seq. ID No. 40) or of the general formula Thr-Tyr-Pro-X-Pro-X-Arg (TYPXPXR) wherein X is a hydrophobic residue. Preferred residues are of the sequence Thr-Tyr-Pro-Ile-Pro-Ile-Arg (TYPIPIR) (Seq. ID No. 41), Thr-Tyr-Pro-Ile-Pro-Phe-Arg (TYPIPFR) (Seq. ID No. 42), and Thr-Tyr-Pro-Val-Pro-His-Arg (TYPVPHR) (Seq. ID No. 43).

Peptides which bind Bacillus cereus having sequences Val-Thr-Ser-Arg-Gly-Asn-Val (VTSRGNV) (Seq. ID No. 100) and consensus peptides of the formula Ser-Pro-Leu-X₁-X₂-His wherein X₁ is His or Arg and X₂ is Arg or Lys (SPLX₁X₂H) were also identified.

The DNA sequence coding for the exemplified peptides is shown. It is clear that other codons that code for the same amino acid may be substituted using codon tables provided in molecular biology treatises.

The inventive method requires mixing phage from the phage display library with spores, incubating the mixture at about room temperature and separating the phage-spore complexes by centrifugation. The phage-spore complexes are washed several times in buffer. The phage was then eluted from the phage-spore complexes with cold buffer at low pH, then quickly neutralized to prevent phage killing. The phage can then be amplified by infecting an appropriate organism. (E. coli was used in the instant case.) The cell lysate obtained from the culture may then be subjected to previous steps repeatedly. After about four rounds, individual clones are purified from the eluted phage. Phage plaques (about 30 were used) were amplified, the genomic DNA extracted and the DNA sequence of the 7-mer and 12-mer peptides encoding region determined. This DNA sequence indicates the sequence of the tight-binding peptide. The indicated protein sequences are tagged and exposed to known spores to determine binding properties.

### DETAILED DESCRIPTION OF THE INVENTION

It is the purpose of this invention to identify short peptide ligands that bind specifically to the spores of microorganisms, particularly those of Bacillus species. The peptide ligands will bind tightly and in a species-specific manner to a physiological or fortuitous receptor on the surface of the spore. This peptide ligand can be used to capture the cognate spore in filters or as part of a detection device (e.g., a capture devise that concentrates the spores for identification by mass spectroscopy, DNA/RNA sequence evaluation, etc.) The peptide ligands can also be used directly in detection/identification devices and procedures. The peptides can be coupled to detectable (e.g., fluorescent, phosphorescent, radioactive, etc.) tags and the peptide-tag conjugates mixed with a sample which can contain cognate spores. If spores are present, they will be bound by the peptide-tag, thereby marking the spores for detection by whatever detector is appropriate for the particular tag. The peptides could also be used antigens in the preparation of vaccines against pathogenic spore-forming bacteria.

Spores of primary interest in the studies disclosed herein were spores of Bacillus subtilis, Bacillus antracis, Bacillus cereus, and Bacillus thuringiensis. However, methods of the invention may also be used to identify and capture other pathogens such as Clostridia species. B subtilis is a target primarily because it is used as a simulant in the development of detection devices for pathogenic B. anthracis. B. antracis is a key target because of its potential as an agent for use in biological warfare and terrorism. B. cereus and B. thuringiensis are targets because they closely resemble B. anthracis and because they are widely distributed in the environment. Thus, they can, potentially, produce false positive readings in detection devices and systems used to identify B. anthracis spores.

Phage Display ligand screening was employed using a commercially available (New England BioLabs) combinatorial library of 2×10⁹ random peptide sequences (7-mer and 12-mer peptides were studied) were individually displayed on the surface of the filamentous coliphage M13. The random peptides were fused to the amino terminus of the minor coat protein PIII. The library is made by inserting a random nucleotide sequence at the beginning of the pIII gene of many copies of the M13 genome. These recombinant genomes are used to produce M13 phage. Each recombinant pIII gene produced a random peptide-pIII fusion protein and five copies of this fusion protein are displayed at one end of the mature phage particle. Thus, the random peptide sequence is displayed at the amino terminus of each pIII copy for a given phage. Furthermore, the random peptide sequence displayed by a particular phage clone can be readily determined by sequencing the peptide-encoding region of the phage genome.

Variations in the process would be known to one of skill in the art. Some of the modifications which enhance productivity are provided herein.

While the methods of the invention were first practiced targeting B. subtilis, then targeting B. anthracis, Bacillus thuringiensis and B. cereus, the methods disclosed herein, particularly the biopanning methods, may be used to identify useful sequences for binding to surfaces of other microorganisms.

The peptides of the invention may be prepared by means known in the art. These peptides can be synthesized, for example, using solid-phase synthesis and standard F-MOC chemistry. Then one would screen the compound using the methods described herein and comparable methods known in the art.

Materials and Methods:

Peptides of interest were identified using a phage display ligand screening system. A phage display peptide library kit (New England BioLabs) was used according to instructions of the manufacturer in the identification process. The phage display library contains random 7-mer peptides (2×10⁹ sequences) fused to the minor coat protein (pIII) of the filamentous coliphage M13. The phage containing the peptide ligands of interest were isolated from the phage library by several cycles of biopanning. The ligands of interest were then identified by sequencing the appropriate genomic region of the isolated phage.

In the biopanning procedure 10¹¹ phage were mixed with 10⁹ spores, incubated for 10 minutes at room temperature, and phage-spore complexes were separated by centrifugation at 4° C. The complexes were washed ten times with ice-cold TBST [50 mM Tris-HCl (pH 7.5), 150 mM NaCl, 0.5% Tween 20] and phage were eluted from the complex with ice-cold elution buffer (0.2 M glycine-HCl, pH 2.2). The phage-containing eluate is immediately adjusted to a pH of about 7. This phage population is amplified by infecting E. coli, and the resulting lysate is used for a second round of biopanning. After about four rounds of biopanning, the eluted phage were plated to obtain single plaques, which were used to prepare large amounts of the particular phage. DNA was extracted from each phage preparation, and the genomic sequence encoding the 7-mer peptide was determined. The DNA of over thirty independent phage isolates of B. subtilis were sequenced and thirteen unique sequences were identified. (See table 1.) All encoded peptides contained amino terminal sequence Asn-His-Phe-Leu. Although the sequences at positions five through seven are not identical, there is a clear preference for certain amino acids.

TABLE 1
Nucleotide and Amino Acid Sequences from
B. subtilis Spore-Binding Phage

| Iso- | | |
|---|---|---|
| late | | |
| 1 | AAT CAT TTT TTG ATT AAG CCG | (Seq. ID No. 2) |
| 2 | AAT CAT TTT TTG AGG TCT CCG | (Seq. ID No. 3) |
| 3 | AAT CAT TTT CTG CCT CGT TGG | (Seq. ID No. 4) |
| 4 | AAT CAT TTT CTT CCT AAG GTG | (Seq. ID No. 5) |
| 5 | AAT CAT TTT CTG TTG CCG CCG | (Seq. ID No. 6) |
| 6 | AAT CAT TTT TTG CCT CCT CGG | (Seq. ID No. 7) |
| 7 | AAT CAT TTT CTG CCT ACT GGG | (Seq. ID No. 8) |
| 8 | AAT CAT TTT CTG ATG CCG AAG | (Seq. ID No. 9) |
| 9 | AAT CAT TTT CTT AAG GGG ACG | (Seq. ID No. 10) |
| 10 | ATT CAT TTT TTG CCG CAG AAT | (Seq. ID No. 11) |
| 11 | ATT CAT TTT CTT CTT TGG CGT | (Seq. ID No. 12) |
| 12 | AAT CAT TTT CTG ATT AGG AAG | (Seq. ID NO. 13) |
| 13 | AAT CAT TTT CTG CCC ACT GCT | (Seq. ID No. 14) |
| 1 | Asn His Phe Leu Ile Lys Pro | (Seq. ID No. 19) |
| 2 | Asn His Phe Leu Arg Ser Pro | (Seq. ID No. 20) |
| 3 | Asn His Phe Leu Pro Arg Trp | (Seq. ID No. 21) |
| 4 | Asn His Phe Leu Pro Lys Val | (Seq. ID No. 22) |
| 5 | Asn His Phe Leu Leu Pro Pro | (Seq. ID No. 23) |
| 6 | Asn His Phe Leu Pro Pro Arg | (Seq. ID No. 24) |
| 7 | Asn His Phe Leu Pro Thr Gly | (Seq. ID No. 25) |
| 8 | Asn His Phe Leu Met Pro Lys | (Seq. ID No. 26) |
| 9 | Asn His Phe Leu Lys Gly Thr | (Seq. ID No. 27) |
| 10 | Asn His Phe Leu Pro GLn Asn | (Seq. ID No. 28) |
| 11 | Asn His Phe Leu Leu Trp Arg | (Seq. ID No. 29) |
| 12 | Asn His Phe Leu Ile Arg Lys | (Seq. ID No. 30) |
| 13 | Asn His Phe Leu Pro Thr Ala | (Seq. ID No. 31) |

For purposes of this application, discussions relating to the particular peptides will refer to the isolate numbers at the left side of the table. The Seq. ID No.'s relate to the computer-readable print-out which must be provided to the various patent offices.

To confirm that the B. subtilis peptides are tight-binding ligands, the following experiment was performed. 10⁷ phage of isolate #4 (NHFLPKV) (Seq. ID No. 15) and 10¹⁰ phage containing random 7-mer sequences were mixed with 10⁹ spores. This mixture was subjected to a single round of biopanning. The eluted phage were plaque-purified and genomic DNA was sequenced as described above. Seven of the ten phage examined contained the sequence of isolate #4. Thus, there was a 700-fold enrichment of this phage, clearly indicating that the isolate #4 peptide bound tightly to the spore.

Attempts to bind the spores of B. subtilis with the 4-mer peptide NHFL (Seq. ID No. 1) showed that sequence to be a poor ligand. However, the 5-mer sequence NHFLP (Seq. ID No. 39) showed tight binding.

In a search of the Swiss-Prot data base of characterized peptides for proteins containing the sequence NHFLP, seven proteins with this sequence were identified. Five are eukaryotic proteins and two are B. subtilis proteins. The first B. subtilis protein is SpsC (Database accession number BG10611), which contains the NHFLP sequence near its amino terminus (i.e., MVQKRNHFLPYSLP-) (Seq. ID No. 16). SpsC appears to be involved in the synthesis of polysaccharides on the surface of the spore. It is probable that this protein uses its amino terminus to attach to a receptor on the spore surface. The instantly claimed peptide ligands may bind to the same site. The second B. subtilis protein is UvrC (Database accession number BG10349), an exonuclease involved in DNA repair. The NHFLP sequence is found in the middle of UvrC, which contains 598 amino acids. Because UvrC is known to be cytoplasmic, a connection between this protein and the peptide ligands is not obvious.

Alternatively, differential display can be utilized to quickly find small molecule analogs or antagonists of present peptides (Greenwood, et al., Multiple display of foreign peptides on a filamentous bacteriophage: Peptides from plasmodium falciparium circumsporozoite protein as antigens. J. Mol. Biol. 206:821-827, 1991).

There were only thirteen unique DNA sequences (out of a total of thirty) from B. subtilis spore-binding phage found. The frequency with which a particular sequence is found may directly reflect the tightness of binding of the encoded peptide. Although the sequences at positions five through seven are not identical, there is a clear preference for certain amino acids. Nearly one-third of all residues in positions 5, 6 and 7 are prolines (12/39), 31% are positively charged (5/39 Arg and 4/39 Lys), and the rest are hydrophobic or hydroxyl-containing. At position five, there is a strong preference for proline (6/13). Thus, it appears that these peptides bind to the same receptor on the spore coat.

The biopanning experiment described above was repeated using a library containing larger 12-mer peptides seen below:

| | ------------------------------------------ | |
|---|---|---|
| 1. | Asn His Phe Leu Lys Ser Gln Pro Gly Val Val Thr | (Seq. ID No. 80) |
| 2. | Asn His Phe Leu Asn Arg Pro Ala Gln Ser Gln Val | (Seq. ID No. 81) |
| 3. | Asn His Phe Leu Pro Pro Lys Met Gly Pro Thr Asp | (Seq. ID No. 82) |
| 4. | Asn His Phe Leu Pro Glu Pro Arg Leu Val Met Pro | (Seq. ID No. 83) |
| 5. | Asn His Phe Leu Ala Pro Gln Pro Pro Val Lys Pro | (Seq. ID No. 84) |
| 6. | Asn His Phe Leu Met Pro Asn Pro Leu Leu Ala Met | (Seq. ID No. 84) |
| 7. | Asn His Phe Leu Ile Pro Pro Glu Pro Leu Arg Glu | (Seq. ID No. 85) |
| 8. | Asn His Phe Leu Pro Leu Asn Pro Pro Ala Pro Ser | (Seq. ID No. 86) |

When the 12-mer peptides were compared with the 7-mer peptides, it appeared that no improvement occurred as a result of using the longer peptides.

Selected peptides were analyzed for tight binding to the spore. Two peptides were synthesized initially: NHFLPKVGGGC (Seq. ID 16) and LFNKHVPGGGC (Seq. ID 17). The first has the amino-terminal sequence of peptide #4 plus a Gly₃ linker and a carboxy-terminal Cys. The second has a randomized sequence using the amino acids of peptide #4 plus the Gly₃ linker and carboxy-terminal Cys. The goal was to label these peptides at the carboxy-terminus with phycoerythrin and examine binding of test and control peptides by fluorescence microscopy and FACS sorting.

Initially, peptide-phycoerythrin conjugates were used for FACS. The advantage is that the conjugates are multivalent and the fluorescence characteristics are well suited for FACS. In some instances, peptides are first being reduced with tris(2-carboxyethyl)phosphine (TCEP) before conjugating with the phycoerythrin. Labeling with smaller fluorochromes such as monovalent 5-iodoacetamido-fluorescein is being used as an alternative.

In order to identify the receptor that interacts with the peptide, biotin-containing cross-linking agent that has been attached to a tight-binding peptide. Cross linkers examined included sulfosuccinimidyl-2-[6-(biotinamido)-2-(p-azidobenzamido)-hexanoamido]ethyl-1′c3′-dithiopropionate)sulfo-SBED). The molecule contains three different functional groups or arms. One arm consists of a biotin handle that can be used for purification using immobilized avidin. Another arm includes a sulfo-NHS (N-hydroxy-succinimido) ester that provides amine coupling capability. When mixed with a tight-binding peptide, NHFLPKV plus GGGC (Seq. ID No. 99) extension, the cross linker is covalently coupled to the peptide through the ε-amino group of the carboxy-terminal lysine residue, with the release of N-hydroxy-succinimide. To assure coupling only through the E-amino group of the lysine, the amino terminus of the peptide (i.e., the α-amino group of asparagine) is temporarily protected. The third arm contains a photosensitive phenyl azide that can be activated by exposure to UV light at wave lengths greater than 300 nm. The activated phenyl azide reacts with nucleophiles, especially amines, in the target molecule.

Once the peptide-cross-linker conjugate was prepared, it was mixed with spores for 10 minutes in the dark to allow peptide-receptor interaction. The complexes were exposed to UV (365 nm) light for 15 minutes at 0C to allow cross-linking to the receptor. The spores were then collected by centrifugation, resuspended in SDS-PAGE loading dye (4% SDS, 10% B-mercaptoethanol, 1 mM dithiothreitol, 125 nM Tris-HCl (pH 6.8), 10% glycerol and 0.05% bromophenol blue) and boiled for 8 minutes to solubilize spore coat proteins (including receptor) and to reduce the disulfide bond that attaches the peptide to the cross-linking agent. Intact spores were removed by centrifugation. The supernatant containing solubilized proteins was dialyzed (MW cutoff: 2000 Da) against phosphate-buffered saline (PBS). The sample was passed over a monomer avidin column and washed with PBS to remove proteins lacking a biotin-containing cross-link. The bound protein/receptor was eluted with PBS containing 2 mM biotin. The fraction containing eluted protein (measured by OD₂₈₀) was dialyzed against H₂O and analyzed by SDS-PAGE. If one or more proteins were detected, they were analyzed by sequencing their amino terminus.

Biopanning with the heptamer phage display library was used to identify tight-binding peptides on the surface of B antracis spores. The spores were prepared from the avirulent delta-Ames strain of the organism (lacking the toxin-encoding plasmid pOX1) and were sterilized by gamma-irradiation by Diagnostics Systems Division of the U.S. Army Medical Research Institute of Infectious Disease, Fort Detrick, Maryland.

Four rounds of biopanning were performed in the manner described above. The genomic DNA of amplified eluates from each round were sequenced directly and genomic DNA of 27 single plaques from the fourth round amplified elute were also sequenced. In the fourth round of biopanning, the definite decrease in titer of the supernatant and the increase in the titer of the eluate indicated the selection of tight-binding phages. The DNA sequences of the amplified eluates from biopanning rounds one through three did not show a change in the randomized 21 bp region. However, the DNA sequence of the amplified eluate from round 4 revealed a strong high-intensity shift and indicated a predominant DNA sequence. Reading the most intense bands in the 7-mer peptide encoding region gave the sequence TSQNVRT (Seq. ID No. 40).

The DNA sequences from single plaques of the fourth round of biopanning are summarized in Table 2. Thirteen of the 27 sequences were the same as the dominant sequence found in the amplified eluate. Two other closely related sequences TYPIPIR (Seq. ID No. 41) and TYPIPFR (Seq. ID No. 42) were represented three times each. Another sequence TYPVPHR (SEQ. ID No. 43) similar to the previous two sequences was found once. The three last sequences define a tight binding sequence of the consensus formula TYPX₁PX₂R wherein X defines hydrophobic residues and where the preferred X₁ is valine (V) or isoleucine (I) and X₂ is isoleucine (I), Phenylalanine (F) or Histidine (H). A listing of the sequences is shown in Table 2. has been shown that the first four amino acids of the most common sequence (TSQN) (Seq. ID 44) is present in domain 3 of the B. anthracis protective antigen.

Sequences 4, 5, 6, and 9 are preferred sequences for binding the spore coat.

TABLE 2
Nucleotide and Amino Acid Sequences from
B. anthracis Spore Binding Phage

Iso-
late

| | | |
|---|---|---|
| 1 | AAT AGT GTT ACT CTT GAG CCG | (Seq ID No. 60) |
| (1) | Asn Ser Val Thr Leu Glu Pro | (Seq ID No. 50) |
| 2 | AAG CCG AGG CAG CCG GGT TTG | (Seq. ID No. 61) |
| (1) | Lys Pro Arg Gln Pro Gly Leu | (Seq. ID No. 51) |
| 3 | TCT ACT CCG GCG TGG CTG TCG | (Seq. ID No. 62) |
| (1) | Ser Thr Pro Ala Trp Leu Ser | (Seq. ID No. 52) |
| 4 | ACT AGT CAG AAT GTG CGG ACG | (Seq. ID No. 63) |
| (13) | Thr Ser Gln Asn Val Arg Thr | (Seq. ID No. 40) |
| 5 | ACT TAT CCT ATT CCG ATT CGT | (Seq. ID No. 64) |
| (3) | Thr Tyr Pro Ile Pro Ile Arg | (Seq. ID No. 41) |
| 6 | ACT TAT CCT ATT CCG TTT CGT | (Seq. ID No. 65) |
| (3) | Thr Tyr Pro Ile Pro Phe Arg | (Seq. ID No. 42) |
| 7 | TCT TAT CCT CAT GGT CAG ATT | (Seq. ID No. 66) |
| (1) | Ser Tyr Pro His Gly Gln Ile | (Seq. ID No. 53) |
| 8 | TTT ACT GGG ACT CTT AAT CCT | (Seq. ID No. 67) |
| (1) | Phe Thr Gly Thr Leu Asn Pro | (Seq. ID No. 54) |
| 9 | ACT TAT CCG GTG CCG CAT CGG | (Seq. ID No. 68) |
| (1) | Thr Tyr Pro Val Pro His Arg | (Seq. ID No. 43) |
| 10 | CGG ACT CCT TCG CTT CCT AGT | (Seq. ID No. 69) |
| (1) | Arg Thr Pro Ser Leu Ser Pro | (Seq. ID No. 55) |
| 11 | TTT AGT GTT CCT CGT ATG CCG | (Seq. ID No. 70) |
| (1) | Phe Ser Val Pro Arg Met Pro | (Seq. ID No. 56) |

The number in ( ) refers to the number of phage containing the sequence.

Studies with B cereus T were undertaken using methods described above. After the fourth round of biopanning, individual phages were cloned. Twenty-two phage cones were picked, the genomic DNA prepared from each, and the DNA coding regions sequenced. The results revealed 8 unique DNA sequences. (See Table 3.) A heptapeptide VTSRGNV (Seq. ID No. 100) having tight-binding properties was identified. This sequence emerged from the pooled genome sequence of amplified phage following the third round of biopanning. The following unique DNA sequences were identified:

TABLE 3
B. cereus T spore tight-binding peptides:

| | | |
|---|---|---|
| 1. | ACG CAT CGT TTG CCT TCT CGG | (Seq. ID No. 101) |
| (2) | Thr His Arg Leu Pro Ser Arg | (Seq. ID No. 110) |
| 2. | GTT ACT AGT AGG GGG AAT GTT | (Seq. ID No. 102) |
| (13) | Val Thr Ser Arg Gly Asn Val | (Seq. ID No. 111) |
| 3. | AAG CTG TGG GTG ATT CCT CAG | (Seq. ID No. 103) |
| | Lys Leu Trp Val Ile Pro Gln | (Seq. ID No. 112) |
| 4. | TAT TCG CCT CCT CAT AGG CAT | (Seq. ID No. 104) |
| | Tyr Ser Pro Leu His Arg His | (Seq. ID No. 113) |
| 5. | TCG TAT CCT CCG TAT TTT GAT | (Seq. ID No. 105) |
| | Ser Tyr Pro Pro Tyr Phe Asp | (Seq. ID No. 114) |
| 6. | CTT TTG TCG CCT CTG CAT CGT | (Seq. ID No. 106) |
| (2) | Leu Leu Ser Pro Leu His Arg | (Seq. ID No. 115) |
| 7. | TTT GAT TCT CCG CTT CGT CGG | (Seq. ID No. 107) |
| | Phe Asp Ser Pro Leu Arg Arg | (Seq. ID No. 116) |
| 8. | TGG TCG CCG CTG CAT AAG CAT | (Seq. ID No. 108) |
| | Trp Ser Pro Leu His Lys His | (Seq. ID No. 117) |

-----------------------------------------------

One DNA sequence (Seq. ID No. 111), found in 13 of the 22 phage, encoded the previously identified tight-binding peptide VTSRGNV. A second sequence was obtained from an inspection of the remaining 7 unique phage DNA sequences. Four of these sequences (4, 6, 7 and 8) contained all or most of the closely related sequence SPL(H or R) (R or K)H. Such results are highly suggestive of a true tight-binding peptide sequence.

A competitive biopanning study was performed using phage displaying unique peptide sequence 8 (WSPLHKH) (Seq. ID No. 117) in this study. 10¹⁰ phage from a random phage library and 10⁷ phage displaying sequence #8 were mixed together, and one round of biopanning was performed using spores of B cereus T. The eluted phage were plaque-purified and genomic DNA was sequenced for twenty phage. Six of the twenty phage contained the sequences of isolate #8. Thus, there was a 300-fold enrichment of this phage, indicating tight binding.

In efforts to find additional tight-binding peptides the wash buffer or wash conditions have been systematically modified. One alteration is the use of either 0.5% or 0.01% Tween 20 and either 3 or 10 washes. These conditions were used in biopanning for B subtilis, wherein number of washes were reduced from 10 to 3. After four rounds of biopanning, an amplified eluted phage pool was sequenced. The results indicate that by changing certain parameters, it is possible to detect new tight binders for some spore species.

It was found that it was possible to precipitate the amplified phage for 30 minutes instead of overnight. Using the abbreviated method, it was possible to omit all titering of phage between the rounds. Using this method, an approximate concentration of amplified phage (1.75×10¹³ pfu/ml) is assumed. Omitting these steps allows a four-round biopanning experiment to be completed in two 12-hour days without affecting results.

Modified versions of the biopanning procedure can also be used wherein phage are permitted to bind spores. Binding complexes are recovered by centrifugation. Complexes are mixed with E. coli to permit phage amplification (under conditions where B. subtilis growth is inhibited), and amplified phage are subjected to additional rounds of biopanning. Tight-binding phage are then recovered by centrifuging spores plus bound phage through a density gradient.

The propensity of the peptides provided herein to bind to spore surfaces makes it possible to capture and identify target bacteria and spores to which the peptides bind. For example, when tagged sequences which bind to the surface of spores of B. subtilis, particularly 5-mer to 12-mer sequences, are placed in the environment believed to contain B. subtilis spores, the presence of the bacteria of interest are identified. Tags such as fluorescent, phosphorescent or calorimetric tags make it possible to visualize the presence of the bacteria. Other tags, such as radioactive tags, may require other equipment such as scintillators to determine the presence or absence of the target organisms. The method described above is particularly useful for identifying contamination of water and food that might cause disease when ingested. Contamination of the air might be established using methods of the invention. The latter is particularly important when the possible contaminant is B. anthracis. It would be possible to attach the peptides identified as having the appropriate binding properties to solid supports to capture spores or spore-forming organisms which bind to the peptide. The particular support will depend on the use. For example, appropriate supports may be natural fibers or polymers which may be in the form of filtering devices, tapes or sponges. Supports having the binding peptides may be used as protective barriers such as masks.

Purified peptides formulated in pharmaceutically acceptable carriers such as buffered saline may be administered to animals in an appropriate amount to elicit an immune response or to bind to the spore to cause alteration in pathogenicity. The method of administration will depend on the organism and the site of infection. Formulations for inhalation may also be buffered to prevent damage to tissue.

Polyclonal antibodies to the sequences of interest can be produced in animals and purified directly from the spleen cells. It is also common to isolate spleen cells from the animal for purposes of producing antibodies. These cells can then be fused with an immortal cell line and screened for monoclonal antibody secretion. Purified antibodies that specifically bind the peptide are within the scope of the present invention. The antibody can be labeled by means generally known in the art using, for example, fluorescent, radioactive or phosphorescent markers, or tags may used in conjunction with a labeled secondary antibody in methods such as ELISA tests. Monovalent, divalent or single chain antibodies can be made which bind the peptides of the invention.

Anti-idiotype antibodies can also be made by means commonly known in the art. Antibodies to the present peptides can exhibit idiotypic mimicry and can be administered to provide protection against bacterial infection. Antibodies to the spore-binding peptides provided herein can be administered to susceptible hosts to block SpsC binding to the spore surface, thus inhibiting development of clinical disease.

What I claim is:

1. A method of obtaining sequences which bind to the surface of a bacterial spore comprising the steps of:
(a) mixing phage from a Phage Display library with spores;
(b) incubating the product of step (a) for sufficient time to allow the phage to complex with the spores;
(c) centrifuging the product of step (b) to obtain the phage-spore complexes;
(d) washing the phage-spore complexes repeatedly;
(e) eluting the phage from the phage-spore complexes with elution buffer;
(f) neutralizing the eluate,
(g) amplifying the eluted phage,
(h) repeating the above steps to perform 3 to 4 rounds of biopanning;
(i) purifying individual clones;
(j) amplifying purified clones, then extracting genomic DNA from each preparation to determine the DNA sequence encoding peptides; and
(k) subjecting the peptides indicated by the DNA sequence to binding studies to determine ability of the peptides to bind to the target spores.

2. A peptide which binds to B. subtilis chosen from peptides of 5-12 amino acids containing the sequence Asn-His-Phe-Leu (Seq. ID No. 1).

3. A peptide of claim 2 containing the sequence Asn-His-Phe-Leu-Pro (Seq. ID No. 39).

4. A peptide which binds to B. anthracis chosen from peptides of the sequences Thr-Ser-Glu-Asn-Val-Arg-Thr (TSQNVRT) (Seq. ID No. 40) or a sequence of the general formula Thr-Tyr-Pro-X-Pro-X-Arg (TYPXPXR) wherein X is a Ile, Val or Leu.

5. A peptide of claim 4 having the sequence TSQNVRT.

6. A peptide of claim 4 having the general formula Thr-Tyr-Pro-X-Pro-X-Arg (TYPXPXR) wherein X is a Ile, Val or Leu.

7. A peptide of claim 6 wherein, in both instances, X is Ile.

8. A peptide of claim 6 wherein, in at least one instance, X is Val.

9. A peptide which binds to B. cereus chose from peptides having the sequence Val-Thr-Ser-Arg-Gly-Asn-Val (VTSRGNV) (Seq. ID No. 100) and Ser-Pro-Leu-X₁-X₂-His wherein X₁ is His or Arg and X₂ is Arg or Lys (SPLX₁X₂H).

10. A composition of matter comprising a peptide ligand which binds with specificity to the surface of a bacterial spore, said ligand being bound to a solid support.

11. A composition of claim 10 wherein the solid support is a polymeric support.

12. A composition of claim 10 wherein the solid support forms a filter.

13. A composition of claim 10 wherein the solid support is a tape or sponge.
